(19) United States
(12) Patent Application Publication
(10) Pub. No.: US 2019/0172566 A1
(43) Pub. Date: Jun. 6, 2019

(54) MOBILE PATIENT-CENTRIC ELECTRONIC HEALTH RECORDS

(71) Applicant: Duke University, Durham (NC)

(72) Inventors: Kevin Schulman, Durham, NC (US); Daniel Chander, Durham, NC (US); Rajan Patel, Durham, NC (US)

(21) Appl. No.: 16/093,318

(22) PCT Filed: Apr. 13, 2017
(86) PCT No.: PCT/US2017/027310

§ 371 Date: Oct. 12, 2018

Related U.S. Application Data
(60) Provisional application No. 62/322,922, filed on Apr. 15, 2016.

(51) Int. Cl. G16H 10/60; G06F 21/62; G06F 16/903

(52) U.S. Cl. CPC G16H 10/60; G06F 16/903; G06F 21/629

(57) ABSTRACT

Systems and methods for processing mobile patient-centric electronic health record data. In one example, the system and method perform or include storing, with a memory in a computing device associated with the patient, a core module associated with the electronic personal health record having personal health information of the patient; assigning, with an electronic processor, permission rights to the personal health information of the patient; receiving a report query for personal health information from a server associated with a service provider; determining the service provider has permission to access the personal health information of the patient; retrieving personal health information from the electronic personal health record in response to determining the service provider has permission to access the personal health information of the patient; and sending the personal health information to the server associated with the service provider.

### CROSS-REFERENCE TO RELATED APPLICATION

This application claims priority to U.S. Provisional Patent Application No. 62/322,922 filed on Apr. 15, 2016, the entire contents of which is herein incorporated by reference.

### BACKGROUND

Goals of health information technology include improving the quality of care and lowering the cost of care. Technology ought to support health information technology use cases to meet these goals. Currently, most health information technology use cases are oriented around providers. For example, there are administrative use cases, such as for patient administration and financial management and clinical use cases, such as electronic health records, laboratory and radiology results, and dispensing medicines in pharmacies.

### SUMMARY

The present disclosure provides, among other things, a data model for a distributed electronic health record (EHR) platform/system that furthers the concept of personalized health care, while accounting for the cloud- and mobile-first society that we live in today. The platform/system 1) has the potential to facilitate cross-provider information exchange at scale, 2) is searchable and accessible, 3) has the potential to maintain or improve on current levels of security and standardization, and 4) enables a novel distributed reporting function for quality assurance and public health. Additionally, the EHR platforms comprise multiple user interfaces, segmented by disease area and for different services. Such systems/platforms will redefine patient experience and supporting health system services.

One of the uses provided by the systems and methods provided herein is to allow patients to have easy access to providers. Additionally, the systems and methods provide for service delivery across the globe including low-resource settings, rather than burdening differing health systems with legacy use cases of existing health information technology.

In one example, the systems and method provide for storing, with a memory in a computing device associated with the patient, a core module associated with the electronic personal health record having personal health information of the patient; assigning, with an electronic processor, permission rights to the personal health information of the patient; receiving a report query associated with the personal health information from a server associated with a service provider; determining the service provider has permission to access the personal health information of the patient; retrieving personal health information from the electronic personal health record in response to the service provider having permission to access the personal health information of the patient; and sending the personal health information to the server associated with the service provider.

One aspect of the present disclosure provides a modular electronic personal health record system that provides patient and provider access to personal health information, and manipulation of such personal health information, on a computing device of a patient, the system comprising, consisting of, or consisting essentially of: (a) a data module comprising one or more data storage systems that comprise personal health information; (b) a service module comprising at least two apps, the apps providing (i) a patient interface allowing the patient to retrieve and/or input personal health information; and (ii) a provider interface allowing a provider to retrieve and/or input personal health information; and (c) a core module that is integrated into the computing device and (i) comprises the personal health records of the patient; and (ii) is capable of interfacing with the data and service modules.

Another aspect of the present disclosure provides a modular electronic personal health record system that provides patient and provider access to personal health information, and manipulation of such personal health information, on a computing device of a patient, the system comprising, consisting of, or consisting essentially of: (a) a core data module comprising one or more extensible data storage modules that comprise the personal health record including personal health information; (b) service modules comprising access from two conceptual perspectives, the patient and the provider (patient app and provider app), the patient apps providing (i) a patient interface allowing the patient to retrieve and/or input personal health information; and (ii) the provider apps allowing a provider to retrieve and/or input personal health information, and (iii) a method of adding extensible data modules to the core data module from the patient apps or the provider apps (c) service modules comprising access to the data modules for reporting purposes, using a federated data management approach; and (d) a core module that is integrated into the computing device and (i) comprises the personal health records of the patient; and (ii) is capable of interfacing with the core data and patient, provider and reporting service modules.

In other embodiments, the data and service modules are stored (as a primary data storage device or as a back-up storage device) on a plurality of servers, each server positioned at a separate location. In yet another embodiment, the servers are connected to the cloud. In another embodiment, the computing device is connected to the cloud. In other embodiments, the services module further comprises at least one 3rd party application for analyzing the personal health information. In other embodiments, the service module comprises a provider interface that allows for the provider to retrieve and/or input personal health information for a plurality of users. In another embodiment, the provider interface further comprises at least one reporting function for the provider that allows for the analysis and reporting of a plurality of personal health information from a plurality of users. In another embodiment, the public health reporting interface further comprises at least one reporting function that allows for the analysis and reporting of a plurality of personal health information from a plurality of users. In yet another embodiment, the service module comprises a patient interface that allows for the patient to retrieve and/or input personal health information. In another embodiment the patient interface further comprises at least one reporting function for the patient that allows for the analysis and reporting of the personal health information.

Another aspect of the present disclosure provides a method for retrieving and analyzing the personal health information of a patient, by the patient, using the system as described herein, the method comprising, consisting of, or consisting essentially of: (a) accessing, by the computing device, the core modules, the core module being able to interface with the data and service modules to store and back-up the patient's personal health information; (b) facilitating the retrieval and analysis of the personal health information by the patient through the patient interface, the retrieval and analysis being facilitated by one or more apps that allow for the review and/or analysis of the personal health information; (c) optionally facilitating input of data from the patient; (d) optionally accepting the inputted data from the patient and incorporating the data into the personal health information; and (e) optionally accepting the inputted data from the patient and incorporating extensible data modules into the personal health record

Another aspect of the present disclosure provides a method for retrieving and analyzing the personal health information of one or more patients by a provider using the system as described herein, the method comprising, consisting of, or consisting essentially of: (a) accessing, by the computing device, the core module(s), the core module being able to interface with the data and service modules to retrieve the one or more patients' personal health information; (b) facilitating the retrieval and analysis of the personal health information by the provider through the provider interface, the retrieval and analysis being facilitated by one or more apps that allow for the review and/or analysis of the personal health information; (c) optionally facilitating input of data from the provider; and (d) optionally accepting the inputted data from the provider and incorporating the data into the personal health information of the appropriate patient.

Another aspect of the present disclosure includes a method for providing public health reporting of health information associated with patients from a physician's office to a health department using embodiments provided herein.

Some embodiments comprise a means of reporting the personal health information, the means comprising at least one reporting function for the patient that allows for the analysis and reporting of the personal health information of the patient. Another embodiment further comprises a means of reporting the personal health information, the means comprising at least one reporting function for the provider that allows for the analysis and reporting of a plurality of personal health information from a plurality of users. Another embodiment comprises a means of reporting the personal health information, the means comprising at least one reporting function for the provider that allows for the analysis and reporting of a plurality of personal health information from a plurality of users.

Another aspect of the present disclosure provides a method for retrieving and analyzing the personal health information of a patient, by the patient, using the system as described herein comprising, consisting of, or consisting essentially of: (a) accessing, by the computing device, the core module, the core module being able to interface with the data and service modules to retrieve the patient's personal health information; (b) facilitating the retrieval and analysis of the personal health information by the patient through the patient interface, the retrieval and analysis being facilitated by one or more apps that allow for the review and/or analysis of the personal health information; (c) optionally facilitating input of data from the patient; and (d) optionally accepting the inputted data from the patient and incorporating the data into the personal health information.

Yet another aspect of the present disclosure provides a method for amending, retrieving and analyzing the personal health information of one or more patients by a provider using the system as described herein, the method comprising, consisting of, or consisting essentially of: (a) accessing, by the computing device, the core module, the core module being able to interface with the data and service modules to retrieve the one or more patients' personal health information; (b) facilitating the retrieval and analysis of the personal health information by the provider through the provider interface, the retrieval and analysis being facilitated by one or more apps that allow for the review and/or analysis of the personal health information; (c) optionally facilitating input of data from the provider; and (d) optionally accepting the inputted data from the provider and incorporating the data into the personal health information of the appropriate patient.

Yet another aspect of the present disclosure provides a method for retrieving and analyzing the personal health information of one or more patients by a public health official using the system as described herein, the method comprising, consisting of, or consisting essentially of: (a) accessing, by the computing device, the core module, the core module being able to interface with the data and service modules to retrieve the one or more patients' personal health information; and (b) facilitating the retrieval and analysis of the personal health information by the public health official through the reporting interface, the retrieval and analysis being facilitated by one or more apps that allow for the review and/or analysis of the personal health information. In some embodiments, the method further comprises a means of reporting the personal health information, the means comprising at least one reporting function for the patient that allows for the analysis and reporting of the personal health information of the patient. In other embodiments, the method further comprises a means of reporting the personal health information, the means comprising at least one reporting function for the provider that allows for the analysis and reporting of a plurality of personal health information from a plurality of users. Yet another aspect of the present disclosure provides all that is disclosed and illustrated herein.

### BRIEF DESCRIPTION OF THE DRAWINGS

The foregoing aspects and other features of the disclosure are explained in the following description, taken in connection with the accompanying drawings, herein:

FIG. 1 illustrates a system for processing mobile patient-centric electronic health records, in accordance with some embodiments.

FIG. 2 illustrates a block diagram of the computing device, in accordance with some embodiments.

FIG. 3 illustrates various software programs stored in the memory shown in FIG. 2, in accordance with some embodiments.

FIG. 4 illustrates a block diagram of the data model showing data flow, in accordance with some embodiments.

FIG. 5 illustrates a flow chart showing how data modules are added to a patient's electronic health record, in accordance with some embodiments.

FIG. 6 illustrates a flow diagram showing the development architecture of the system in accordance with one embodiment.

FIG. 7 illustrates a flow chart of a method for processing mobile patient-centric electronic health records, in accordance with some embodiments.

### DETAILED DESCRIPTION

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

Articles “a” and “an” are used herein to refer to one or to more than one (i.e. at least one) of the grammatical object of the article. By way of example, “an element” means at least one element and can include more than one element.

Unless otherwise defined, all technical terms used herein have the same meaning as commonly understood by one of ordinary skill in the art to which this disclosure belongs.

Rather than adopting a provider-centric or technology-based approach, the systems and methods of the present disclosure begin with a design-based approach to envision how individuals might want to interface with information technology to consume their health care. In doing so, three overarching goals are identified. Patients should have 1) 24/7 access to care, 2) easy & affordable care, and 3) feel empowered to take control of their own health. In achieving these goals for patients utilizing the described systems and methods, individuals should a) be informed about the steps they ought to take in order to improve their health, b) be able to carry their health information with them to another provider anywhere in the world, and c) search their health information and be able to ask questions of the data.

Further, the methods of storing medical information as provided herein have been designed to facilitate reporting to enable provider quality assurance activities, and public quality assurance activities including health reporting.

A common criticism of modern electronic health records in use today is the information overload that physicians must navigate through in order to retrieve/update a single piece of information. In response to this, the applications provided herein will be built with a modular architecture ensuring that only data elements appropriate for the individual patient (or someone designated by the patient, e.g., their family, guardian, etc.) are included as data modules at the individual level.

One aspect of the present disclosure provides a modular electronic personal health record system that provides patient and provider access to personal health information, and manipulation of such personal health information, on a computing device of a patient, the system comprising, consisting of, or consisting essentially of: (a) a data module comprising one or more data storage systems that comprise personal health information; (b) a service module comprising at least two apps, the apps providing (i) a patient interface allowing the patient to retrieve and/or input personal health information; and (ii) a provider interface allowing a provider to retrieve and/or input personal health information; and (c) a core module that is integrated into the computing device and (i) comprises the personal health records of the patient; and (ii) is capable of interfacing with the data and service modules.

Another aspect of the present disclosure provides a modular electronic personal health record system that provides patient and provider access to personal health information, and manipulation of such personal health information, on a computing device of a patient, the system comprising, consisting of, or consisting essentially of: (a) a core data module comprising one or more extensible data storage modules that comprise the personal health record including personal health information; (b) service modules comprising access from two conceptual perspectives, the patient and the provider (patient app and provider app), the patient apps providing (i) a patient interface allowing the patient to retrieve and/or input personal health information; and (ii) the provider apps allowing a provider to retrieve and/or input personal health information, and (iii) a method of adding extensible data modules to the core data module from the patient apps or the provider apps (c) service modules comprising access to the data modules for reporting purposes, using a federated data management approach; and (d) a core module that is integrated into the computing device and (i) comprises the personal health records of the patient; and (ii) is capable of interfacing with the core data and patient, provider and reporting service modules.

Another aspect of the present disclosure provides a modular electronic personal health record system that provides patient and provider access to personal health information, and manipulation of such personal health information, on a computing device of a patient, the system comprising, consisting of, or consisting essentially of: (a) a data module that comprises one or more data storage systems that in turn comprise personal health information; (b) a service module that comprises conceptually at least two apps, the apps providing (i) a patient interface allowing the patient to retrieve and/or input personal health information; and (ii) a provider interface allowing a provider to retrieve and/or input personal health information; and (c) a core module that is integrated into the computing device and (i) comprises the personal health records of the patient; (ii) is capable of being backed up to a patient-designated storage platform, and (iii) is capable of interfacing with the data, service and reporting modules.

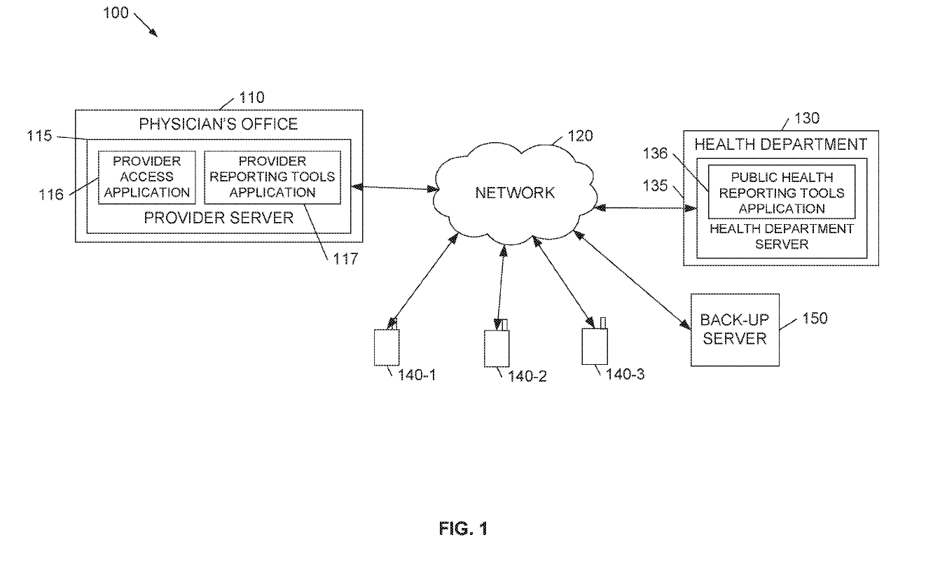

FIG. 1 illustrates a system 100 for processing mobile patient-centric electronic health records, in accordance with some embodiments. In some embodiments, the system 100 includes a physician's office 110, a health department 130, and network 120. Also included in FIG. 1 is a provider server 110 located at the physician's office 110 and a health department server 130 located at the health department 130, a number of computing devices 140-1, 140-2 and 140-3, and a backup server 150 coupled to a network 120 (for example, the internet). In some embodiments, the provider server 110 includes a provider access application 116 and a provider reporting tool application 117. In one example, the provider server 110 is associated with a physician's office. In some embodiments, the health department server includes a public health reporting tool application 136. In one example, the health department server is associated with a health department and is capable of receiving electronic health record information associated with patients associated with computing devices 140-1, 140-2, and 140-3. In some embodiments, the computing device 140 comprises a portable communication device such as a smart phone, a tablet or a PDA.

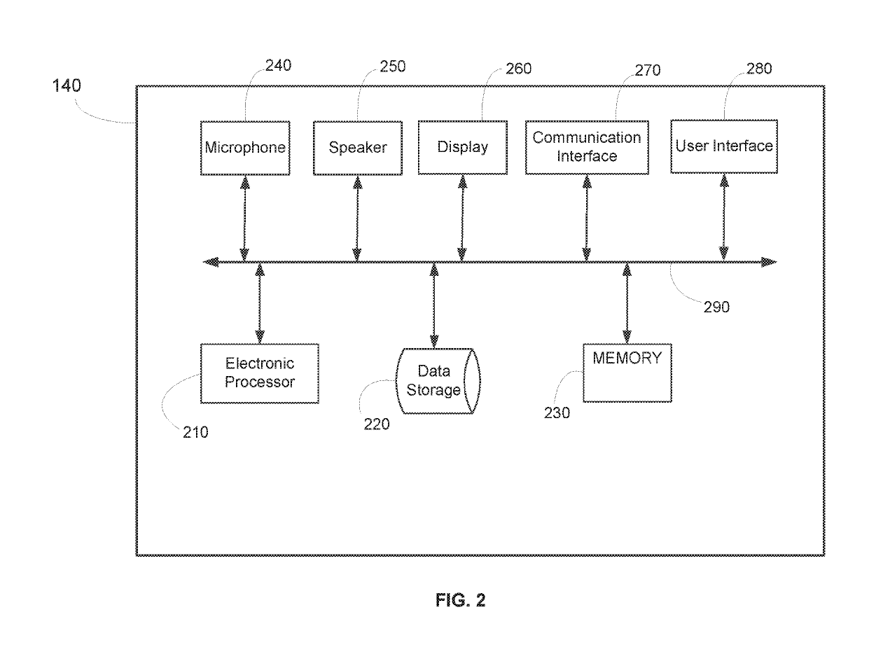

FIG. 2 illustrates a block diagram of the computing device 140, in accordance with some embodiments. The computing device 140 may combine hardware, software, firmware, to implement the method of processing mobile patient-centric electronic health record data as provided herein. In some embodiments, the computing device 100 includes an electronic processor 210, a data storage device 220, a memory 230, a microphone 240, a speaker 250, a display 260, a communication interface 270, a user interface 280 that can be hardware-based (for example, electronic mouse, keyboard, trackball, stylus, touch-pad, touchscreen, etc.) or software-based and a bus 290. In one embodiment, the electronic processor 210 may include at least one microprocessor and be in communication with at least one microprocessor. The microprocessor interprets and executes a set of instructions stored in the memory 230. In some embodiments, the memory 230 includes, for example, random access memory (RAM), read-only memory (ROM), and combinations thereof. In some embodiments, the memory 230 has a distributed architecture, where various components are situated remotely from one another, but may be accessed by the electronic processor 210.

The data storage device 220 may include a non-transitory, machine-readable storage medium that stores, for example, one or more databases. In one example, the data storage device 220 also stores executable programs, for example, a set of instructions that when executed by one or more processors cause the one or more processors to perform the one or more methods describe herein. In one example, the data storage device 220 is located external to the computing device 140.

The communication interface 270 provides the computing device 140 a communication gateway with an external network (for example, a wireless network, the internet, etc.). The communication interface 270 may include, for example, an Ethernet card or adapter or a wireless local area network (WLAN) integrated circuit, card or adapter (for example, IEEE standard 802.11a/b/g/n). The communication interface 270 may include address, control, and/or data connections to enable appropriate communications with the external network.

The user interface 280 provides a mechanism for a user to interact with the computing device 140. As noted above, the user interface 280 includes input devices such as a keyboard, a mouse, a touch-pad device, and others. In some embodiments, the display 260 may be part of the user interface 280 and may be a touchscreen display. In some embodiments, the user interface 280 may also interact with or be controlled by software programs including speech-to-text and text-to-speech interfaces. In some embodiments, the user interface 280 includes a command language interface, for example, a software-generated command language interface that includes elements configured to accept user inputs, for example, program-specific instructions or data. In some embodiments, the software-generated components of the user interface 280 includes menus that a user may use to choose particular commands from lists displayed on the display 260.

The bus 290, or other component interconnection, provides one or more communication links among the components of the computing device 140. The bus 290 may be, for example, one or more buses or other wired or wireless connections. The bus 290 may have additional elements, which are omitted for simplicity, such as controllers, buffers (for example, caches), drivers, repeaters, and receivers, or other similar components, to enable communications. The bus 290 may also include address, control, data connections, or a combination of the foregoing to enable appropriate communications among the aforementioned components.

In some embodiments, the electronic processor 210, and the memory 230 are included in a single computing device (for example, within a common housing), such as a smart telephone, smart watch or other wearable, or another suitable computing device. In these embodiments, the electronic processor 210 executes a software program that is locally stored in the memory 230 of the computing device 140 to perform the methods described herein. For example, the electronic processor 210 may execute the software program to access and process data (for example, images) stored in the memory 230 and/or the data storage device 220. Alternatively or in addition, the electronic processor 210 may execute the software application to access data (for example, images) stored external to the computing device (for example, on a server accessible over a communication network such as the internet). The electronic processor 210 may output the results of processing the accessed data (for example, an automatically created narrative of the images) to the display 260 included in the computing device 140.

In some embodiments, the electronic processor 210 may be included in the server or another device that communicates with the server over a wired or wireless network or connection. For example, in some embodiments, the electronic processor 210 may be included in the server and may execute a software application that is locally stored on the server to access and process data as described herein. In particular, the electronic processor 210 may execute the software application on the server, which a user may access through a software application, such as a browser application or a mobile application) executed by a computing device of the user. Accordingly, functionality provided by a system 100 as described below may be distributed between a computing device 140 of a user and a server remote from the computing device 140. For example, a user may execute a software program on his or her personal computing device to communicate with another software program executed by an electronic processor included in a remote server.

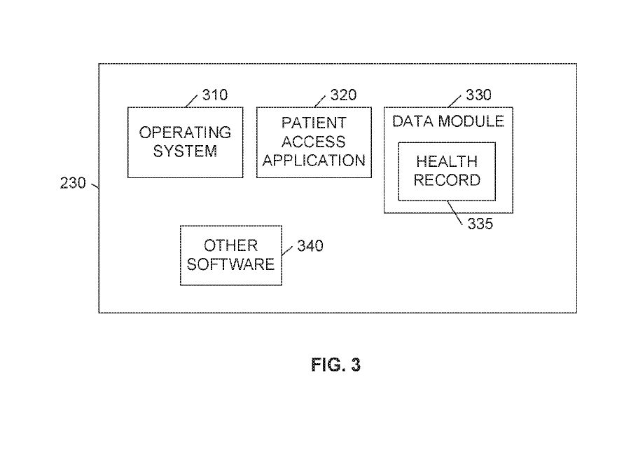

FIG. 3 illustrates various software programs stored in the memory 230 shown in FIG. 2, in accordance with some embodiments. In some embodiments, the memory 230 includes an operating system 310, a patient access application 320, a data module 330 including a health record 335, and other software 340.

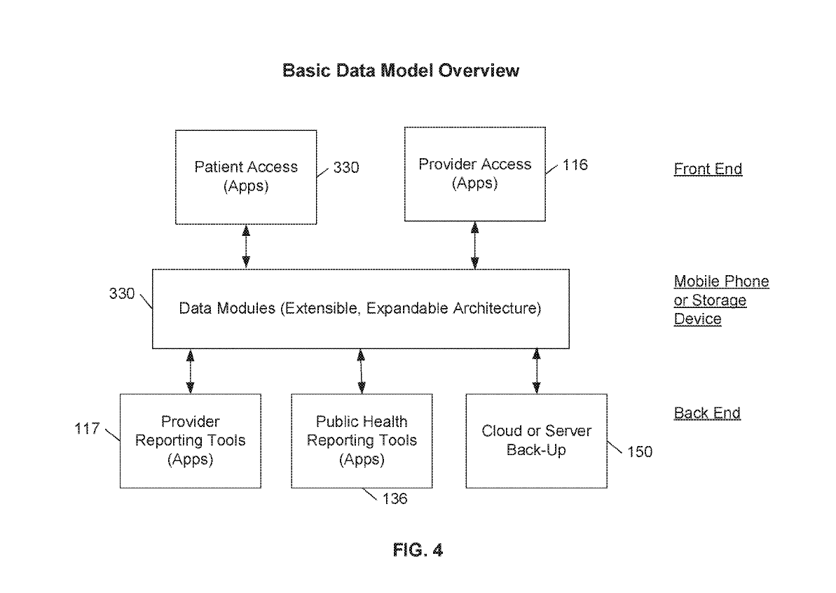

FIG. 4 illustrates a block diagram of the data model showing data flow, in accordance with some embodiments. As shown in FIG. 4, data modules 330 may be accessed by patient access applications 330, provider access applications 116, provider reporting tools 117, public health reporting tools 136 and a backup server 150. In some embodiments, the data module includes a core module including minimal information, for example a health record ‘skeleton’. In some embodiments, the health record ‘skeleton’ contains all the pieces of information that are universally applicable to patient health (such as patient identification). Other patient-specific health information can be pulled from data/service repositories or added by the patient or by the physician as needed. The complexity of the application scales with the complexity of the patient's suite of illnesses but never delivers excessive amounts of information. The data modules and services are defined and explained in the following paragraphs.

In certain aspects, the systems comprise a core module. As used herein, the term “core module” refers to a type of data module (see ‘Data Module’) that is automatically integrated into the application upon an initial download. The core modules make up the health record skeleton. These modules are deemed relevant for most, if not all, patients and providers. The minimization of core modules keeps the application extremely lightweight and customizable but their presence in the architecture also enhances out-of-the-box usability of our applications.

In another aspect, the systems comprise a data module. As used herein, the term “data module” refers to a schema for data that is relevant to a common topic (e.g. patient identification, asthma, vaccinations). The view layer comprises an interface with which an individual can create their own schema and assign the desired provisions/restrictions to it. Upon submission, the module will be reviewed and, if approved, added to the Global Data Module Repository (GDMR). Whoever has access to the module (doctor/provider, patient, etc.) in the GDMR will be able to add it to their app.

The systems further comprise a service module. As used herein, the term “service module” or “service” refers to a mini-application. The view layer will contain an interface with which an individual can create their own service, using our 3rd party integrations and proprietary set of tools, to improve user experience, help clinician workflow, and/or improve health outcomes. Upon submission, the module will be reviewed and, if approved, added to the Global Service Repository (GSR). Individuals with the relevant access privileges will be able to add it to their app. In some embodiments, the service modules further comprise at least one 3rd party application for analyzing the personal health information. In another embodiment, the service modules comprise a provider interface that allows for the provider to retrieve and/or input personal health information for a plurality of users.

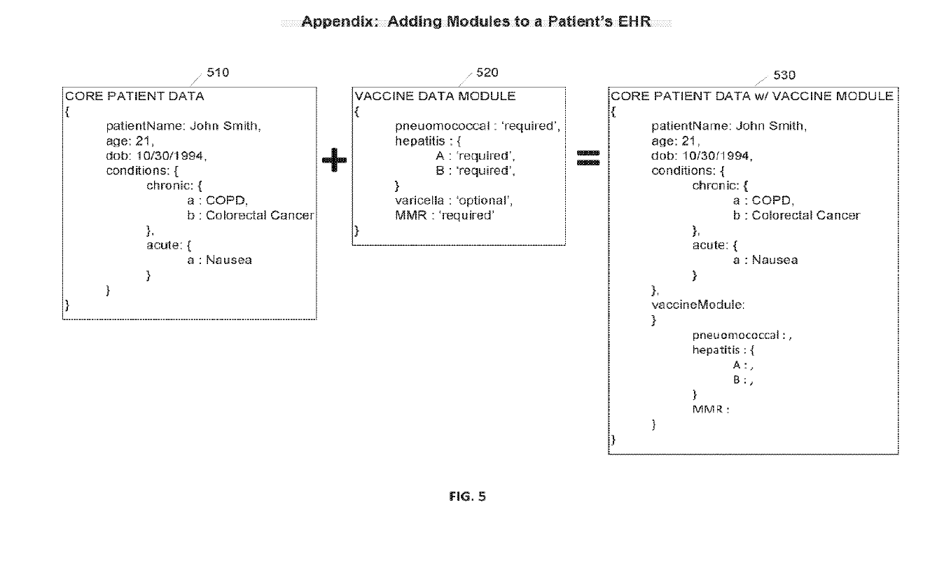

FIG. 5 illustrates an example how data modules are added to a patient's EHR in accordance with the systems and methods of the present disclosure. In the example shown, the core patient data module 510 maybe added to a vaccine data module 520 to generate a combined module 530 that includes both the core patient data module 510 and the vaccine data module 520.

Architectural Design.

Any architectural design may be utilized in the systems and methods provided herein. In some embodiments, the systems and methods utilize Representational State Transfer (REST) architecture described by Roy Thomas Fielding in 2000 at https://www.ics.uci.edu/˜fielding/pubs/dissertation/fielding dissertation.pdf and incorporated by reference herein. The primary benefit of using a REST architecture is scalability. Because the server and client are loosely coupled, the server will not be overburdened when the number of concurrent users grows. In some embodiments, one or more application programming interfaces (APIs) are used to communicate between the various software components described herein. In one example, data between the computing device 140 and one or more entities connected using the network 120 is retrieved and sent using application programming interfaces. Additionally, this architecture will enable developers and users to perform Create, Read, Update, Delete operations on the application program interface (API), which will facilitate the customizable, modular approach we hope to adopt for our platform.

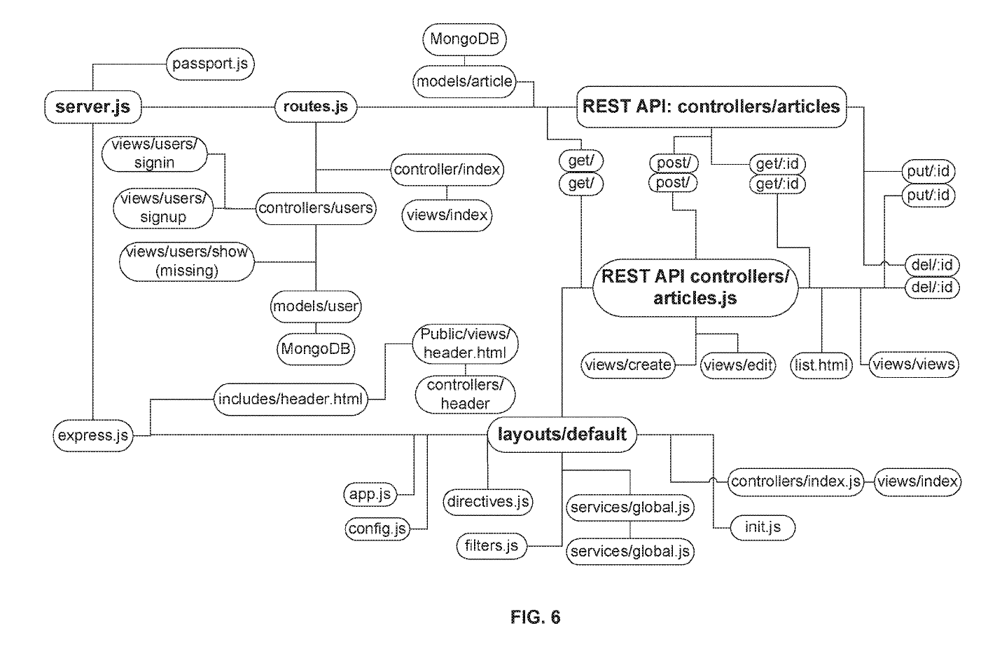

FIG. 6 illustrates a flow diagram showing the development architecture of the system in accordance with one embodiment. FIG. 6 illustrates how programming languages and design principles are woven together, creating the framework for an application that is poised for high-speed scalability and facilitates the modular application structure desired in accordance with one embodiment of the present disclosure.

Data interchanging (sending data between the server and client) is a core functionality of EHR Platform/systems of the present disclosure. The two main formats that facilitate data interchanging are JSON and XML. In some embodiments, JSON is used because (i) it is lightweight and transmits data more quickly than XML, and (ii) it integrates well with the full JavaScript architecture (see “Programming Language Specification”) provided herein.

Programming Language Specification.

The systems described herein may utilize any database architecture that is suitable for housing and organizing EHR. In some embodiments, the database utilized in the systems and methods provided herein comprises noSQL databases such as MongoDB. The benefits of using a noSQL database such as MongoDB for our EHR Platform, over the currently used Relational Database Management Systems (RDBMS), are threefold:

No-Typical-Patient Mentality. RDBMSs utilize rigid tabular structures to store data, resulting in a system where irrelevant information is stored about a patient. MongoDBs non-relational document structure enables the possibility of eliminating extraneous data.

User Centricity. RDBMSs store patient data across hundreds of data tables, which are linked together via keys. This makes it extremely difficult for a user to access their data. A non-relational database will enable our platform to create schemas where the patient is the common factor, and not a specific attribute or condition.

Incorporation into the Modularized Data Model according to one embodiment of the present disclosure. With MongoDB, providers and other module-creating stakeholders will be able to customize the modules that they create so that they are most useful to the community of patients that they treat.

Further support for utilizing the noSQL database format is because healthcare data has been recently considered to be more appropriate for a NoSQL form of database rather than the traditional RDBMS, for the following reasons below, as adapted from Ercan et al; 2014:

Data Size: With the size of healthcare data increased over time, data size becomes a bottleneck for EHR systems. NoSQL databases are based on horizontal scalability, which allows for easy and automatic scaling.

Multi-modal Data: With multi-modal healthcare data, such as free-text notes, images, and other complex data, flexible data models offered by NoSQL databases allow semi or unstructured data to be stored easily in comparison with RDBMS.

Data Availability: Healthcare data should always be accessible for continuity of healthcare services. NoSQL databases provide high availability due to distributed nature and replication of data.

Data Adding: Healthcare data is normally added, not updated. Eventual consistency suggested by NoSQL database architecture is considered acceptable for most use cases.

Data Sharing: Healthcare data sharing requires EHR access from multiple locations—a high performance system necessary to respond to data access request in timely manner. NoSQL databases offer higher performance than relational databases in many use cases.

The frontend and backend needs of the systems provided herein can be met with several languages, including but not limited to, AngularJS, NodeJS, ExpressJS and the like. In some embodiments, the EHR platform described herein comprises Angular.js and Node.js, both implementations of JavaScript. Since MongoDB is implemented in JavaScript, use of Angular.js and Node.js allows for an almost seamless integration. While Angular.js is the leading frontend technology today, Node.js is only adopted by a small (but growing) community of developers. In recognition of this, a transition to a Java backend is also a possibility within the scope of the present disclosure.

In some embodiments, the electronic health record data may be stored and accessed using a blockchain based access-control manager. Blockchain is a decentralized peer-to-peer architecture with nodes consisting of network participants. Each member (for example a patient, a physician or a service provider, a health department, etc.) in the network stores an identical copy of the blockchain and contributes to the collective process of validating and certifying digital transactions for the network. Members in the distributed network record digital transactions into a shared ledger. To add transactions, members in the network run algorithms to evaluate and verify the proposed transaction. If a majority of the members in the network agree that the transaction is valid, the new transaction is added to the shared ledger. Changes to the shared ledger is reflected in all copies of the blockchain in a very short time. Since all the members in the network have a complete copy of the blockchain no single member has the power to tamper or alter data.

Data Storage and Ownership.

A patient's health record resides on their computing device (e.g., a mobile device such as a phone), ensuring that health information can be effectively transported between health facilities. The underlying data, including all of the patients' health records and all data modules and services, will be stored on the mobile phone or mobile storage. The data can be backed-up and retrieved in the event of a failure from the cloud or other storage technology, by utilizing a distributed network of servers across the country. In some embodiments, the MapReduce model will be used when accessing data to maximize fault tolerance. In other words, distributed computing will enable users and other data-accessing entities to use and search their data even if one (or several) servers in the country are offline or damaged.

In some embodiments, the data and service modules are stored and/or backed-up on a plurality of servers, each server positioned at a separate location. In another embodiment, the servers are connected to the cloud. In yet another embodiment, the computing device is connected to the cloud.

There is currently significant debate as to who should be the owner of health data. Popular opinion in the United States is that the health information belongs to the patient while the health record is the property of the provider. In our view, the patient should own their health information and their health record. In some embodiments, access rights to health record of a patient can be granted to physicians and other third parties at the discretion of the patient. Given the societal benefits of aggregated patient data, from clinical trial research to public health efforts, patients should be encouraged to share their data with society.

Core Services & Use Cases.

The core services of such a technology platform would be the Data Modules, the Illness-Specific Data Modules, the front end services: patient access services (apps), the provider access services (apps), and the back end services: provider reporting tools, public health reporting tools, and the cloud or server back-up (FIG. 1).

Some embodiments of use cases include, at the patient-level, a shared personal health plan with his/her care provider and other care givers, shared care groups, interactive educational modules with click through requirements and quizzes.

Other use cases involve the development of federated reports for the provider, such as monthly patient health summary reports or comparative averages for metrics such as blood pressure changes for all patients, could also be implemented. Federated reports are those that are generated by sending a report query to each individual's personal health record (the phone or mobile device data modules) to record the specific data elements from the query, and then only extracts the data that are subject to the query from the individual's record. The results of individual queries are then aggregated into a report at the provider level (ie., in a Federated reporting structure, the entirety of the data modules are not aggregated into an analysis file; only the data from the queries are aggregated into an analysis file).

Federated reports on the level of the public health entity (ministries of health) could help pinpoint the unique IDs of individuals during specific outbreaks (e.g. having the information of those who were not vaccinated transmitted to the relevant providers in the event of a measles outbreak) and also be potentially valuable.

In some embodiments, the provider interface further comprises at least one reporting function for the provider that allows for the analysis and reporting of a plurality of personal health information from a plurality of users.

In some embodiments, the public health interface further comprises at least one reporting function that allows for the analysis and reporting of a plurality of personal health information from a plurality of users.

In some embodiments, the service module comprises a patient interface that allows for the patient to retrieve and/or input personal health information. In some embodiments, the patient interface further comprises at least one reporting function for the patient that allows for the analysis and reporting of the personal health information.

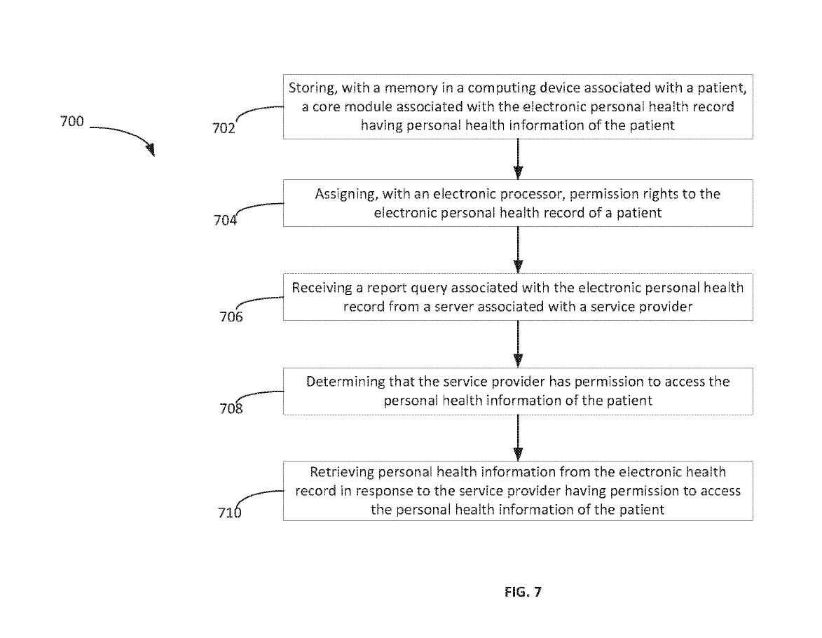

FIG. 7 illustrates a flow chart of a method 700 for processing mobile patient-centric electronic health records, in accordance with some embodiments. At block 702, the method 700 includes storing, with the memory 230 in the computing device 140 associated with a patient, a core module associated with the electronic health record having personal information of the patient. At block 704, the method 700 includes assigning, with the electronic processor 210, permission rights to the electronic personal health record of the patient. At block 706, the method 700 includes receiving a report query associated with the personal health information from a server 115 associated with the service provider. At block 708, the method 700 includes determining that the service provider has permission to access the personal health information of the patient. At block 710, the method 700 includes retrieving personal health information from the electronic health record in response to the service provider having permission to access the personal health information of the patient.

Another aspect of the present disclosure provides a method for retrieving and analyzing the personal health information of a patient, by the patient, using the system as described herein comprising, consisting of, or consisting essentially of: (a) accessing, by the computing device, the core module, the core module being able to interface with the data and service modules to retrieve the patient's personal health information; (b) facilitating the retrieval and analysis of the personal health information by the patient through the patient interface, the retrieval and analysis being facilitated by one or more apps that allow for the review and/or analysis of the personal health information; (c) optionally facilitating input of data from the patient; and (d) optionally accepting the inputted data from the patient and incorporating the data into the personal health information.

Yet another aspect of the present disclosure provides a method for retrieving and analyzing the personal health information of one or more patients by a provider using the system as described herein, the method comprising, consisting of, or consisting essentially of: (a) accessing, by the computing device, the core module, the core module being able to interface with the data and service modules to retrieve the one or more patients' personal health information; (b) facilitating the retrieval and analysis of the personal health information by the provider through the provider interface, the retrieval and analysis being facilitated by one or more apps that allow for the review and/or analysis of the personal health information; (c) optionally facilitating input of data from the provider; and (d) optionally accepting the inputted data from the provider and incorporating the data into the personal health information of the appropriate patient.

In some embodiments, the method further comprises reporting the personal health information with a means comprising at least one reporting function for the patient that allows for the analysis and reporting of the personal health information of the patient.

In other embodiments, the method further comprises reporting the personal health information with a means comprising at least one reporting function for the provider that allows for the analysis and reporting of a plurality of personal health information from a plurality of users.

In other embodiments, the method further comprises reporting the personal health information with a means comprising at least one public health reporting function for that allows for the analysis and reporting of a plurality of personal health information from a plurality of users.

Any patents or publications mentioned in this specification are indicative of the levels of those skilled in the art to which the invention pertains. These patents and publications are herein incorporated by reference to the same extent as if each individual publication was specifically and individually indicated to be incorporated by reference. In case of conflict, the present specification, including definitions, will control.

One skilled in the art will readily appreciate that the present invention is well adapted to carry out the objects and obtain the ends and advantages mentioned, as well as those inherent therein. The present disclosure described herein are presently representative of preferred embodiments, are exemplary, and are not intended as limitations on the scope of the invention. Changes therein and other uses will occur to those skilled in the art which are encompassed within the spirit of the invention as defined by the scope of the claims.

What is claimed is:

1. A computing device associated with a patient, the computing device comprising:
a memory including a core module to store an electronic personal health record having personal health information of the patient; and
an electronic processor connected to the memory and configured to
assign permission rights to the personal health information of the patient,
receive a report query for personal health information from a server associated with a service provider,
determine whether a service provider has permission to access the personal health information of the patient,
retrieve personal health information from the electronic personal health record in response to determining whether the service provider has permission to access the personal health information of the patient, and
send the personal health information to the server associated with the service provider.

2. The computing device of claim 1, wherein the core module includes one or more extensible data storage modules.

3. The computing device of claim 1, wherein the report query is received from a provider access application.

4. A modular electronic personal health record system, the system comprising:
a computing system including an electronic processor configured to
store in a core module an electronic personal health record having personal health information of a patient, the core module including one or more extensible data storage modules;
assign, with the electronic processor, permission rights to the personal health information of the patient;
receive a report query for personal health information from a server associated with a service provider;
determine the service provider has permission to access the personal health information of the patient;
retrieve personal health information from the electronic personal health record in response the service provider having permission to access the personal health information of the patient; and
send the personal health information to the server associated with the service provider.

5. The system of claim 4, further comprising a service module.

6. The system of claim 5, wherein the service module includes a patient access application.

7. The system of claim 5, wherein the service module includes a provider access application.

8. The system of claim 7, wherein the report query is received from the provider access application.

9. The system of claim 7, wherein the provider access application allows a provider to retrieve and/or input personal health information of the patient.

10. The system of claim 4, further comprising a means for reporting the personal health information, the means comprising at least one reporting function for the patient that allows for the analysis and reporting of the personal health information of the patient.

11. The system according to claim 10, wherein the data module and the service module are stored on a plurality of servers, each server positioned at a separate location.

12. The system according to claim 4, wherein the service module further comprising at least one third party application for analyzing the personal health information.

13. The system according to claim 4, wherein the service module comprises a provider interface configured to allow the service provider to retrieve and/or input personal health information for a plurality of users.

14. The system according to claim 13, wherein the provider interface further comprises at least one reporting function for the service provider that allows for the analysis and reporting of a plurality of personal health information from a plurality of users.

15. The system according to claim 5, wherein the service module comprises a patient interface is configured to allow the patient to retrieve and/or input personal health information.

16. The system according to claim 15, wherein the patient interface further comprises at least one reporting function for the patient that allows for the analysis and reporting of the personal health information.

17. A method for processing electronic personal health record of a patient, the method comprising:
storing, with a memory in a computing device associated with the patient, a core module associated with the electronic personal health record having personal health information of the patient;
assigning, with an electronic processor, permission rights to the personal health information of the patient;
receiving a report query associated with the personal health information from a server associated with a service provider;
determining the service provider has permission to access the personal health information of the patient;
retrieving personal health information from the electronic personal health record in response to the service provider having permission to access the personal health information of the patient; and
sending the personal health information to the server associated with the service provider.
